(19) United States
(12) Patent Application Publication
(10) Pub. No.: US 2015/0133454 A1
(43) Pub. Date: May 14, 2015

(54) NANOSTRUCTURED MUCOADHESIVE MICROPARTICLES

(71) Applicant: SNU R&DB FOUNDATION, Seoul (KR)

(72) Inventors: Young Bin Choy, Seoul (KR); Chun Gwon Park, Seoul (KR)

(73) Assignee: SNU R&DB FOUNDATION, Seoul (KR)

(21) Appl. No.: 14/401,398

(22) PCT Filed: May 14, 2013
(86) PCT No.: PCT/KR2013/004252

§ 371 Date: Nov. 14, 2014

(30) Foreign Application Priority Data
May 15, 2012 (KR) 10-2012-0051348
May 13, 2013 (KR) 10-2013-0053851

(51) Int. Cl. A61K 31/498; A61K 47/34; A61K 47/10; A61K 31/382

(52) U.S. Cl. CPC A61K 31/498; A61K 31/382; A61K 47/34; A61K 47/10
USPC 514/249; 514/772; 514/432

(57) ABSTRACT

Provided is a nanostructured mucoadhesive microparticle including a biocompatible polymer, the microparticle having a surface with a nanostructure formed thereon. The present nanostructured mucoadhesive microparticle having an increased retention time on the mucous as well as with a minimized irritation to the surface can be advantageously used as a drug delivery vehicle. Thus the increased bioavailability of a therapeutic agent administered by the present microparticle leads to an increased therapeutic efficacy, reduced dosage and reduced number of administration as well as significant cost savings and improved patient convenience resulted therefrom.

### BACKGROUND OF THE INVENTION

1. Field of the Invention

The present disclosure generally relates to a mucoadhesive technology used for drug delivery systems.

2. Description of the Related Art

Parenteral and non-parenteral drug administration is often hampered by the low drug bioavailability due to a short contact time between the drug and the permeation layer through which the drug is absorbed, resulting from a low residence time of the drug on the permeation layer combined with a rapid drug washout by bodily fluid.

Thus for the effective drug absorption, a higher dose or a repeated administration of the drug is required which causes inconvenience to the patients.

For example, eye drops containing therapeutic agents for treating eye disease are easy to apply and acting relatively rapid. However, it suffers from very low bioavailability due to a tear clearance of the drug which is not apt for sustained delivery and also the low permeability of a drug through an epithelial barrier of the eye surface is problematic. Particularly, the liquid type of eye drops promotes the washout mechanism by tears and thus almost 75% of the eye drops administered is removed immediately after the administration. This leads to a low bioavailability such that just less than 5% of the drug administered is actually utilized (D. Ghate and H.F. Edelhauser, Ocular drug delivery. Expert Opin Drug Deliv 3, 275-287, 2006). Thus the administration of drug using eye drops requires a higher dose and/or a continuous use in which repeated administration of drug 2-3 times a day with a constant time interval is required, which results in a low acceptance by the patients.

U.S. Pat. No. 5,942,243 relates to a mucous adhesive composition for administration of biologically active materials to the animal tissues and discloses a drug delivery system comprising plastic graft copolymer consisting of a polystyrene macro monomer and a hydrophilic acidic monomer. European patent publication No. 1652535 relates to a semisolid mucoadhesive formulation and discloses a drug delivery system through vagina using biomucoadhesive material.

There still exist needs for the development of system which is able to provide a longer residence time to release drug in the mucous membrane in a sustained manner. In such cases, the high bioavailability of drug achieved will increase the patient's acceptance mainly due to the reduced number of administration as well as the significant cost savings resulted therefrom.

### DETAILED DESCRIPTION OF THE INVENTION

### Problems to Be Solved

To solve the problems above the present disclosure is to provide a drug delivery system which can provide an increased residence time on the mucous membrane and a sustained drug release property while having a minimized irritation to the surface of the tissue applied.

### SUMMARY OF THE INVENTION

The present disclosure relates to nanostructured mucoadhesive microparticles comprising a biocompatible adhesive agent, the microparticle having a surface with a nanostructure formed thereon thereby having an enlarged specific surface area and an increased adhesiveness to a mucous membrane.

The present microparticles may further comprises a diffusion control material such as a polylactide, a polyglycolide, a poly(lactic-co-glycolic acid), a polyorthoester, a polyanhydride, a poly(amino acid), a poly(hydroxybutyric acid), a polycaprolactone, a polyalkylcarbonate, an ethylcellulose, a chitosan, a starch, a guar gum, a gelatin, a collagen, or a combination thereof.

The present microparticles have an improved or increased adhesiveness to the mucous membranes for example ocular, pulmonary, buccal, bronchial, endometrium, esophageal, olfactory, penile, vocal, sublingual, rectal, gastric, intestinal, colonic, oral, nasal, anal, or vaginal mucous membrane without being limited thereto.

The present microparticles may be formulated in various forms considering various factors for example such as administration routes, for example, in one embodiment the present microparticles are formulated in a tablet for a topical application.

In one embodiment, the biocompatible adhesive agent is a water-soluble polymer, or water-soluble synthetic polymer or water-soluble cellulose derivative.

In other embodiment, the biocompatible adhesive agent is PEG. In one embodiment, the present microparticles comprises is a PEG as a biocompatible adhesive agent and PLGA as a diffusion control agent.

The present microparticles have nanostructures formed on the surface which may be prepared by electrospinning and freeze-milling the biocompatible adhesive agent.

The present microparticles can be advantageously used for a sustained release of a small molecule, a protein drug, a radionuclide, a nucleic acid based drug, or a combination thereof through the adhesiveness to a mucous membrane.

In one embodiment, the present microparticles are applied to an ocular mucous membrane, and the microparticles further comprise as a therapeutic agent for treating ocular disease, for example including an antiviral agent, an antibacterial agent, an anti-fungal agent, an antiallergic agent, an nonsteroidal anti-inflammatory agent, an anti-inflammatory agent, an anti-inflammatory-analgesic agent, an anti-inflammatory enzyme agent, an antibiotic, a sulfa agent, a synthetic penicillin, a therapeutic agent for treating glaucoma, a therapeutic agent for treating cataract, a miotic, a mydriatic, a topical astringent, a vasoconstrictor, an agent for preventing rise of intraocular pressure, a therapeutic agent for treating ocular hypertension, a topical anesthetic, an α1-blocker, a β-blocker, a β-blocker, a carbonic anhydrase inhibitor, a topical selective H1-blocker, an adrenal cortical hormone, a vitamin B12, a coenzyme type vitamin B2, an anticholinesterase agent, or an organic iodine preparation.

In other aspect, the present disclosure further relates to a mucoadhesive system for a drug delivery comprising the nanostructured mucoadhesive microparticle according to the present disclosure.

In one embodiment, the present system can be advantageously used for drug delivery particularly for a sustained drug delivery.

The foregoing summary is illustrative only and is not intended to be in any way limiting. Additional aspects and/or advantages of the invention will be set forth in part in the description which follows and, in part, will be obvious from the description, or may be learned by practice of the invention.

### Advantageous Effects

According to one or more embodiments of the present invention, it is an advantage of the present invention that the mucoadhesive drug delivery system of the present disclosure has overcome the disadvantages associated the conventional system such as a low bioavailability of the drug due to its rapid wash out by tears. The present nanostructured mucoadhesive microparticle having an increased retention time on the mucous layer and a sustained drug release property as well as with a minimized irritation to the surface can be advantageously used as a drug delivery vehicle. The increased bioavailability of a therapeutic agent administered by the present microparticle leads to an increased therapeutic efficacy, reduced dosage and reduced number of administration as well as significant cost savings and improved patient convenience resulted therefrom.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1A is a schematic representation of preparing (a) nanostructured microparticles (NM) and (b) their tablet formulation. A tablet embedded with the NM was prepared using a porous PVA matrix. To prepare the NM, a solution of PLGA or a blend of PLGA and PEG was electrospun to give a nanofibrous sheet, which was then freeze-milled by a steel impactor shuttling back and forth at 14 cycles·s-1 at −196° C. (6770 Freezer Mill, Spex, Metuchen, N.J., USA). To prepare a dry tablet embedded with the NM, an aqueous PVA solution (2% w/v in pH 7.4 PBS) suspended with the NM was added into a mold, which was then freeze-dried for 1 day.

FIG. 1B is scanning electron micrographs of MM comprising biocompatible polymer fabricated according to one embodiment of the present disclosure, in which (a) PLGA MS (microsphere), (b) PLGA/PEG MS, (c) PLGA NM and (d) PLGA/PEG NM. The insets show the microparticle of each type in a higher magnification. MS of (a) and (b) fabricated using the conventional emulsion method has a smooth surface while NM has a rough surface, which resulted in an increased specific surface area compared to MS. The scale bars=20 μm.

FIG. 1C is scanning electron micrographs of a nanofibrous sheet produced using (a) PLGA or (b) a blend of PLGA and PEG in the process of fabricating NM in one embodiment of the present disclosure, in which the sheet was sputter coated with platinum for 10 minute and the image was taken using a scanning electron microscope (SEM; 7401 F, Jeol, Japan). The scale bars=20 μm.

FIG. 2A is a graph of the size distribution profile of PLGA/PEG MS and PLGA/PEG NM according to one embodiment of the present disclosure, which shows a similar distribution pattern between the MS and NM.

FIG. 2B is a table showing the size and the amount of Nile Red loaded on NM and MS of FIG. 2A, in which it was found that the mean size of the four types of microparticle is 2 micrometer and the amount of Nile Red loaded is 8 microgram/ml.

FIG. 3 is analysis results to confirm the presence or absence of PLGA and PEG within NM, in which (a) is a graph of GPC (Gel permeation chromatography) data from intact PLGA, PEG and the four different types of microparticles and (b) is a numerical representation of the graph of (a) which shows that the peak retention volume of PLGA MS and PLGA NM was identical to that of the intact PLGA indicating that the PLGA MS and PLGA NM fabricated is only composed of intact PLGA. While the retention volume of PLGA/PEG MS and PLGA/PEG NM had two peaks, each corresponding to that of PLGA and PEG respectively, indicating that both PLGA/PEG MS and PLGA/PEG NM contain PLGA and PEG.

FIG. 4 is analysis results to confirm the amount of PEG within microparticle, in which (a) represents intact PLGA, TGA data of intact PEG, (b) represent TGA data of the four different microparticles, which shows that PLGA MS and PLGA NM is composed of 100% PLGA consistent with GPC data while PLGA/PEG MS and PLGA/PEG NM is composed of about 90% PLGA and about 10% of PEG.

FIG. 5 is fluorescence micrographs of a dry tablet embedded PLGA/PEG NM containing Nile Red in (a) the top view and (b) the side view and SEM images of the surfaces of the tablets containing (c) PLGA/PEG MS and (d) PLGA/PEG NM and Fluorescence micrographs of (f) PLGA/PEG MS and (g) PLGA/PEG NM suspended in pH 7.4 PBS. The scale bars=20 μm.

FIG. 6 is a graph showing the retention time of the microparticles in the preocular region of the rabbit eye, in which the four different types of nanostructured microparticles according to one embodiment of the present disclosure was delivered to the rabbit eyes using two different types of formulation.

FIG. 7 is fluorescence images of Nile-Red loaded microparticles remaining on the preocular surface of the rabbit eyes. The images were taken at a specified time after the administration of each of the four different (a) suspensions and (b) tablets. The black and white arrows indicate the locations of the eyeball and the exposed lower fornix of the rabbit eye, respectively. The scale bars=5 mm.

FIG. 8 is a result of cytotoxicity test using PLGA/PEG NM according to one embodiment of the present disclosure indicating no-cytotoxicity by the present NM (cell cytotoxicity%=˜0.8%).

FIG. 9 is a result of safety test using PLGA/PEG tablet of the present disclosure administered on the rabbit eye, in which images were obtained (a) before the administration, (b) at 1 hour, (c) at 2 hour and (c) at 24 hour after the administration. The scale bars=5 mm. During the test, no significant side effect was found except a minor conjunctival injection. Conjunctivitis was found in the normal eye that was left untreated, which was thought to result from the dryness of the eye left open during the anesthesia. The Intra ocular pressure (IOP) was not significantly changed after the administration of PLGA/PEG NM tablet. The IOP measured was 16.8±1.4 mmHg, which is a normal value found in rabbits.

FIG. 10A is a drug release data from PVA tablet (no microparticle), and PLGA MS, PLGA/PEG MS, PLGA NM and PLGA/PEG NM loaded with Brimonidine .

FIG. 10B is a drug release data from PVA tablet (no microparticle), and PLGA MS, PLGA/PEG MS, PLGA NM and PLGA/PEG NM loaded with Dorzolamide.

FIG. 11A is a result showing the decrease in IOP over time in the rabbit eye after administration of formulation loaded with Brimonidine, in which * represents that IOP was statistically significantly decreased at 6, 7, 8, 9, 10, 11 and 12 hours after administration of PLGA/PEG NM tablet compared to control and other formulation (p<0.05).

FIG. 11B is a result showing the decrease in IOP over time in the rabbit eye after administration of formulation loaded with Dorzolamide, in which * represents that IOP was statistically significantly decreased at 3, 4, 5, 6, 7, 8 and 9 hours after administration of PLGA/PEG NM tablet compared to control and other formulation (p<0.05).

FIG. 12 is a result showing the concentration of brimonidine at aqueous humor of the rabbit over time after the administration of Alphagan P and PLGA/PEG NM tablet., in which * represents that administration of PLGA/PEG NM tablet caused statistically significant difference in the concentration at 1, 1.5, 2, 3, 5 and 7 hours after administration compared to Alphagan P.

FIG. 13 is fluorescence images showing the drug delivery through the intestine using the present nanostructured microparticles. The scale bars=1 cm.

### DETAILED DESCRIPTION OF THE EMBODIMENT

The present disclosure relates to a mucoadhesive microparticle comprising a biocompatible adhesive material and optionally a diffusion control material with a nanostructure formed on the surface of the microparticle.

The term biocompatible refers to one that does not produce an unacceptable or undesirable response in the recipient and is intended to denote a material that upon contact with a living element such as cells or tissues, does not cause toxicity. The biocompatible adhesive material or agent used for the present disclosure adheres to mucin on the mucous membrane and thus increases the retention time of the microparticle of the present disclosure. Further the nanostructure formed on the surface increases the specific surface area of the microparticle, acting synergistically with the adhesiveness to increase the retention time. Also, the nanostructure increases frictional force which also attribute to the increased retention time in the mucous membrane. That is, the present microparticle having an increased retention or residence time on the mucous membrane with a nanostructure formed thereon comprising a mucoadhesive material with or without diffusion control property can be advantageously used as a drug delivery system. An example of the mucoadhesive material without a diffusion control property is chitosan which is not only used as a diffusion-wall material to control the diffusion of a drug but also has a mucoadhesive property.

The term “nanostructure” or “nanoform” refers to a structure the dimension of which is less than 1000 nm, for example about 100 nm, about 10 nm, about 5 nm, which may be formed using various ways. In one embodiment of the present disclosure, the nanostructure may be formed by milling nanofiber sheets or by milling spherical particles to prepare the particles with nanostructures formed on the surface thereon.

The microparticle of the present disclosure has a nanostructure formed on the surface thereof, which as a result increases the specific surface area. This prevents the microparticles from being rapidly washed out from the site applied such as ocular surface due to the increased frictional force with the surface. Also the use of a mucoadhesive material is acting synergistically with the increased specific surface area to increase the adhesiveness of the microparticles to the mucous membrane.

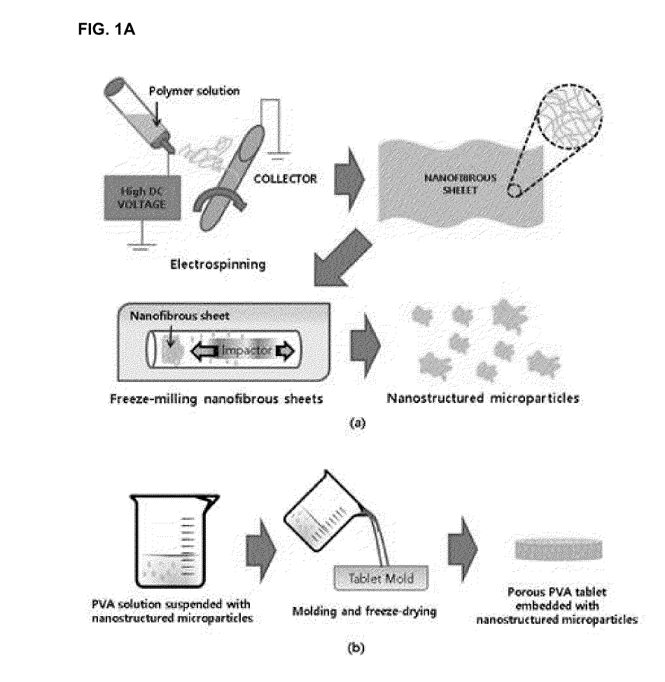

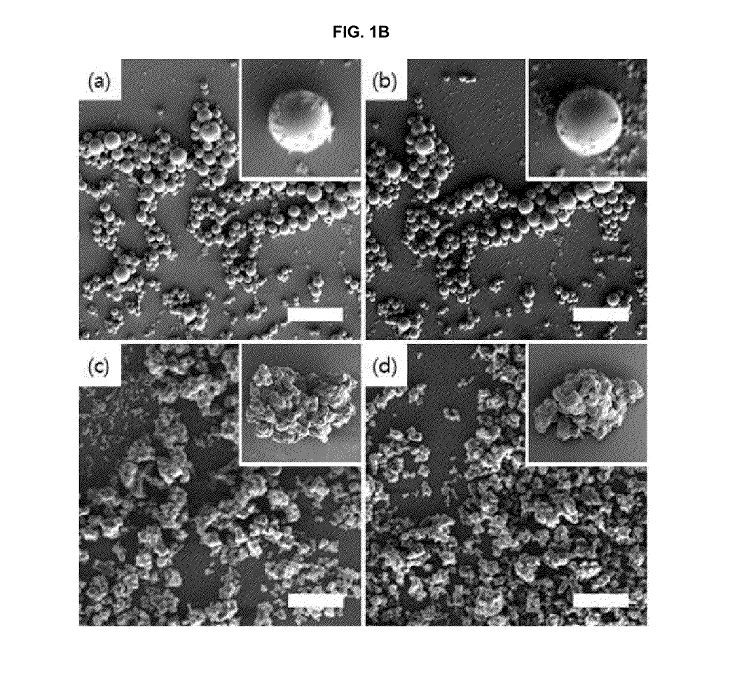

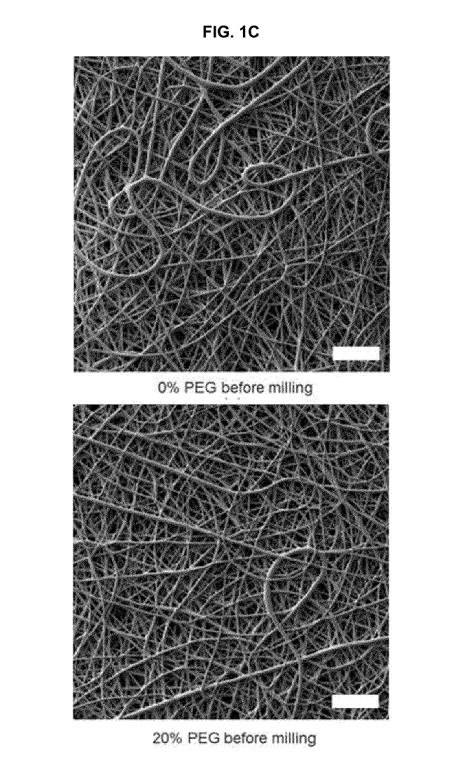

The microparticles with a nanostructure formed on the surface thereof may be prepared using methods known in the art (Dhital et al., Effect of cryo-milling on starches: functionality and digestibility, Food Hydrocolloids, 24, 152-163, 2010) or the method exempliied in FIG. 1. In one embodiment of the present disclosure, freeze-milling method is used

The term “mucous membrane” as used herein is a membrane lining bodily cavities or canals that are exposed to the outside or internal organs and covered with epithelium which is involved in adsorption and secretion. Mucous membranes line many tracts and structures of the body, including the mouth, nose, eyeball, anus, vagina, eyelids, windpipe and lungs, stomach and intestines, ureters, urethra, and urinary bladder and the like. Such membranes includes for example an ocular, pulmonary, buccal, bronchial, endometrium, esophageal, olfactory, penile, vocal, sublingual, rectal, gastric, intestinal, colonic, oral, nasal, anal, or vaginal mucous membrane.

The term “delivery” as used herein refers to a transfer of a desired agent or component over a period of time following administration to a target site. The delivery includes for example a transfer of a therapeutic agent from the microparticles through mucous membranes to a target.

The microparticles of the present disclosure with an increased mucoadhesiveness may be prepared in a variety of dosage forms or formulations. In one embodiment, dry tablets in which tablets of water-soluble polymer embedded with the nanostructured microparticles are used. The dry tablet forms are advantageous for drug delivery over the suspensions without microparticles in which the dry tablet can reduce the loss of drug administered at the early stage and provide the continued therapeutic effect of the agent over extended period of time due to its longer residence time on the mucous membrane.

The biocompatible adhesive or mucoadhesion material which may be used for the present disclosure is an agent that promotes the adhesiveness to the surface, particularly the mucous membranes and includes ones that are known in the art, particularly biocompatible water soluble polymers which are degraded in cells and tissues of the body. The examples of such include, but are not limited to carboxymethylcellulose and polymethacrylic acid based polymers for example polyethyleneglycol, polyacrylic acid, poly-2-hydroxyethylmethacrylic acid and the like. Further examples may be found in Kharenko et al., Pharmaceutical Chemistry Journal Vol. 43, No. 4,pp 200-208 (2009). In one embodiment, PEG is used.

The microparticles of the present disclosure may further comprise a diffusion control agent. In the case in which the adhesives employed also have a diffusion control property, the diffusion control agent may not be used.

The diffusion control agents are used for a sustained release of the therapeutic agent and thus without their use, the therapeutic agent may be released immediately on the ocular mucous membrane by the moisture present on the eye. As such the diffusion control agents that reside on the eye in the form of microparticles increase the residence time of the therapeutic agent resulting in the increase efficacy of the agent. The diffusion control agent should be safe to the tissue or organs it is applied without causing irritation. Particularly when they are used on the eye, the dimension should be around in the order of 10 μM. The example includes but is not limited to polylactide (PLA), polyglycolide (PGA), poly(lactic-co-glycolic acid) (PLGA), polyorthoester, polyanhydride, polyamino acid, polyhydroxybutyric acid, polycaprolactone, polyalkylcarbonate, ethyl cellulose, chitosan, starch, guargum, gelatin or collagen.

In one embodiment, PLGA is used, which is particularly compatible for the present microparticles which need to be biocompatible and not cytotoxic.

The nanostructured microparticles of the present disclosure which have an increased residence time on the mucous membrane are advantageously used as delivery vehicles for a sustained release of a therapeutic agent

The term biologically active ingredients refer to any compound, substance, medicament having a biological activity or function in the body, and which is suitable for administration to a mammal, e.g., a human, more particularly in the context of the present invention, the example of which includes carbohydrates, amino acids, oligopeptides, polypeptides, proteins, oligonucleotides, polynucleotides, nucleic acids, hapten, epitopes, cells, vitamins, and hormones and the like. The term drug or therapeutic agents refer to any compound, substance, medicament having a therapeutic or pharmacological effect, and which is suitable for administration to a mammal, e.g., a human, more particularly in the context of the present invention and used for the treatment, prevention and/or diagnosis of disease.

In other aspect of the present disclosure, the microparticles of the present disclosure adhere or attach to various mucous membranes and are advantageously used as delivery vehicles for a sustained release of therapeutic or biologically active agent such as small molecules, protein drugs, radionuclide, nucleic acid based drug or the combinations thereof.

The drugs or therapeutic agents loaded within the microparticles are suitable for administration to preocular region as a form of eye drops. The nanostructured microparticles of the present disclosure are advantageously used as delivery vehicles for various ocular drugs known in the art.

For example, ocular drugs which may be used for the present disclosure include but are not limited to antiviral agents (keratitis caused by herpes simplex), antibacterial agents (infectious diseases: conjunctivitis, blepharitis, corneal tumor and dacryocystitis), anti-fungal agents, antiallergic agents (allergic conjunctivitis, pollinosis and vernal conjunctivitis), anti-inflammatory agents (conjunctivitis, superficial keratitis, marginal blepharitis and scleritis), nonsteroidal anti-inflammatory agents (allergic conjunctivitis), anti-inflammatory-analgesic agents, anti-inflammatory enzymatic agents (chronic conjunctivitis), antibiotics (infectious diseases: trachoma, conjunctivitis, blepharitis, marginal blepharitis, keratitis, hordeolum, corneal ulcer, tarsadenitis and dacryocystitis), sulfa agents (trachoma, conjunctivitis, blepharitis, marginal blepharitis, corneal ulcer and keratitis), synthetic penicillin (infectious diseases), therapeutic agents for treating glaucoma, therapeutic agents for treating cataract, miotics, mydriatics, topical astringents, vasoconstrictors, agents for preventing rise of intraocular pressure, therapeutic agents for treating ocular hypertension, topical anesthetics, al -blockers (glaucoma and ocular hypertension), β-blockers (glaucoma and ocular hypertension), β1-blockers (glaucoma and ocular hypertension), carbonic anhydrase inhibitors, topical selective H1-blockers (allergic conjunctivitis), adrenal cortical hormone (nosotropic method for inflammatory diseases of external and anterior ocular segments), vitamin B12 (asthenopia), coenzyme type vitamin B2 (keratitis and blepharitis), anticholinesterase agents (glaucoma, accommodative esotropia and myasthenia gravis), organic iodine preparations (central retinitis and the like)

Also the disease that may be treated with such drugs includes for example, eye infection, allergic conjunctivitis, pollinosis and vernal catarrh and the like.

The drugs known in the art include but are not limited to acyclovir, acitazanolast hydrate, azulene, anthranilic acid, ascorbic acid, amlexanox, isopropyl unoprostone, idoxuridine, ibudilast, indomethacin, epinephrine, erythromycin, lysozyme chloride, apraclonidine hydrochloride, oxybuprocaine hydrochloride, carteolol hydrochloride, cyclopentolate hydrochloride, dipivefrin hydrochloride, cefmenoxim hydrochloride, dorzolamide hydrochloride, pilocarpine hydrochloride, phenylephrine hydrochloride, bunazosin hydrochloride, betaxolol hydrochloride, befunolol hydrochloride, levocabastine hydrochloride, levobunolol hydrochloride, lomefloxacin hydrochloride, ofloxacin, carbachol, dipotassium glycyrrhitinate, glutathione, sodium cromoglycate, chloramphenicol, hydrocortisone acetate, prednisolone acetate, cyanocobalamin, diclofenac sodium, distigmine bromide, homatropine hydrobromide, silver nitrate, naphazoline nitrate, calcium diiodostearate, sulfisoxazole, sulbenicillin sodium, dexamethasone, tobramycin, tranilast, tropicamide, nipradilol, norfloxacin, pimaricin, pirenoxine, ketotifen fumarate, pranoprofen, flavin-adenine dinucleotide, fluorometholone, predonisolone, bromofenac sodium hydrate, pemirolast potassium, helenien, timolol maleate, miopin, dexamethasone sodium m-sulfobenzoate, ecothiopate iodide, latanoprost, lidocaine hydrochloride, atropine sulfate, gentamicin sulfate, sisomicin sulfate, dibekacin sulfate, micronomicin sulfate, dexamethasone sodium phosphate, betamethasone disodium phosphate, levofloxacin

In other aspect, the present disclosure relates to a mucoadhesive system for drug delivery comprising the nanostructured microparticles according to the present disclosure. The present mucoadhesive systems increase the residence time at the site applied and thus are suitable for a sustained release of the drug loaded within the NM targeting various mucous membranes

The present disclosure is further explained in more detail with reference to the following examples. These examples, however, should not be interpreted as limiting the scope of the present invention in any manner.

### EXAMPLES

### Materials and Methods

Poly (lactic-co-glycolic acid) (PLGA; 50:50; i.v=0.43 dl/g) and polyethylene glycol (PEG; average MW=6 kDa) were purchased from Lakeshore Biomaterials (AL, USA) and Acros Organics (NJ, USA), respectively. Polyvinyl alcohol (PVA; average MW=31−50 kDa, 87%-89% hydrolyzed) and Nile Red were obtained from Sigma (MO, USA). Dichloromethane (DCM) and acetone were supplied from JT Baker (NJ, USA). Dimethylformamide (DMF), trahydrofurane (THF) and phosphate-buffered saline (PBS; pH 7.4) were obtained from Mallinckrodt (MO, USA), Daejung (Korea) and Seoul National University Hospital Biomedical Research Institute (Seoul, Korea), respectively. Proparacaine hydrochloride (Alcaine; 0.5% ophthalmic solution) was purchased from Alcon-Couvreur (Puurs, Belgium). Ketamine hydrochloride (Ketamine), xylazine (Rompun) and acepromazine maleate (Sedaject) were obtained from Yuhan (Seoul, Korea), Bayer (Leverkusen, Germany) and Samu Median (Yesan, Korea), respectively.

### Example 1

### Preparation of Microsphere (MS) and Nanostructured Microparticles (NM)

To evaluate the effect of the microparticle morphology and materials that promoting the mucoadhesiveness of the microparticles on the retention time on the preocular surface, 4 different microparticles, i.e., PLGA MS: PLGA/PEG MS: PLGA NM and PLGA/PEG NM were prepared as described below.

Fluorescent dye Nile Red was added to the microparticles prepared for a quantitative analysis. Specifically 4-5 mg of microparticles was dissolved in 50 ml of acetone under vigorous stirring for 1 hr followed by a quantitative measurement using a fluorimeter (FS2, Scinco, Korea) as previously described (Dutta, A. K. et al., J Photochem Photobiol A Chem 1996, 93, 57-64).

1-1 Preparation of MS (Spherical microparticles)

MS was prepared as described before (Tadros, T. et al., Adv Colloid Interface Sci 2004, 108-109, 303-18). Briefly, either 500 mg PLGA or a blend of 500 mg PLGA and 100 mg PEG was dissolved in 5 ml DCM, where 5 mg Nile Red was also dissolved as a marker. The resulting solution was then dispersed in an aqueous solution of PVA (20 ml; 1% w/v) and sonicated at 100 W for 5 s (Model 5 Digital Sonic Dismembrator, Fisher Scientific, PA, USA). The emulsion was then added in 80 ml of an aqueous solution of 1% w/v PVA and stirred at 100 rpm under vacuum (−12.5 psi) for 30 min to evaporate the solvent.19 The suspension was filtered (nylon net filter, 11-μm pore, Millipore, Billerica, Mass.) to obtain the MS smaller than 10 μm, which were washed thoroughly with DI water and freeze-dried.

### 1-2 Preparation of NM

To fabricate the nanostructured microparticles (NM), either 90 mg PLGA and or a blend of 90 mg PLGA and 18 mg PEG was dissolved in 0.3 ml of the solvent mixture of DCM, DMF and THF (3:1:1=v/v/v), where 0.9 mg Nile Red was also dissolved as a marker. The resulting solution was then electrospun for 30 min under the following conditions (Nano NC, Korea) to obtain the nanofibrous sheets: applied voltage, 20 kV; collector distance, 10 cm; flow rate; 0.6 ml/h.47 The sheets were then freeze-milled (6770 Freezer Mill, Spex, Metuchen, N.J., USA) at −196° C. for 60 min.30 The resulting particles were suspended in an aqueous solution of PVA (100 ml; 1% w/v) and stirred at 100 rpm under vacuum (-12.5 psi) for 30 min, which was intentionally conducted to have the NM exposed to the same condition as with the MS in emulsion. The suspension was then filtered (nylon net filter, 11-μm pore, Millipore, Billerica, Mass.) to obtain the NM smaller than 10 μm. The particles were then washed thoroughly with DI water and freeze-dried.

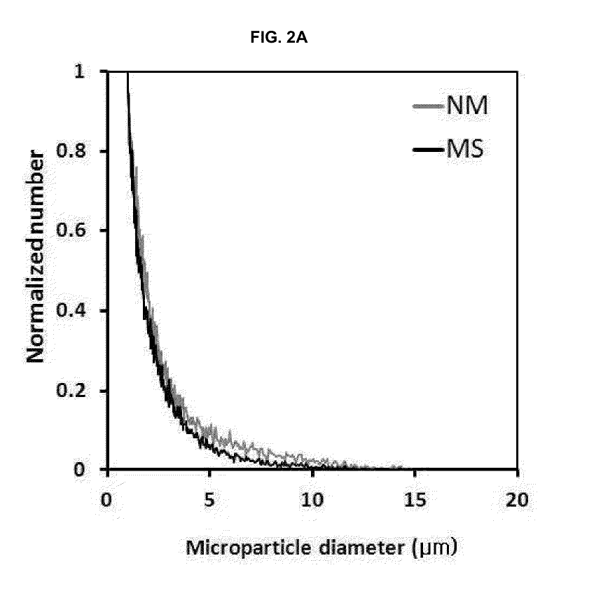

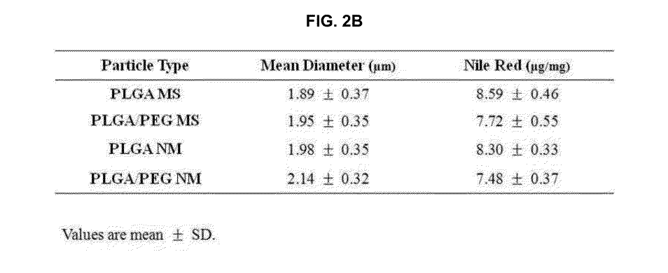

Results are shown in FIGS. 1 and 2. MS (microsphere) prepared using PLGA and a blend of PLGA/PEG as shown in (a) and (b), respectively of FIG.1B exhibits a round shape with smooth surface. In comparison, Nanostructured microparticles (NM) prepared by electrospinning of PLGA and a blend of PLGA/PEG followed by freeze-milling as shown in (c) and (d), respectively of FIG. 1B exhibits a rough surface due to the nanostructure formed thereon thus having a large surface area. That is, MS has a smooth surface in contrast to NM which has a rough surface composed of agglomerated nanofibers.

PEG that was initially added in the amount of 20 wt % did not appear to influence the microparticle morphology.

To prevent the irritation to the preocular region, only the microparticles of <10 μm in size were collected for use by filtering and used for the test. The mean diameter of the microparticles prepared (PLGA MS, PLGA/PEG MS, PLGA NM, PLGA/PEG NM) is shown in FIG. 2. As shown in FIG. 2, the mean diameter was measured to be 1.8-2.2 μm. The microparticles in this size range (<10 μm) were expected to minimize possible eye irritation and allow particle clearance through the lacrimal canals which is 300-500 μm in diameter.

### Example 2

### Preparation of Microparticle Formulations

The microparticles were formulated into two distinct dosage forms, aqueous suspension and a dry tablet. To prepare a suspension, 0.5 mg microparticles were homogeneously dispersed in 30 μl PBS (pH 7.4). To prepare a dry tablet, a 30 μl drop of 2% w/v PVA in pH 7.4 PBS was suspended with 0.5 mg microparticles, which was then added into a mold (6.5 mm in width, 3.5 mm in length, 2.5 mm in height) and freeze-dried for at least 6 hours.

Total of 8 different formulations were prepared: PLGA MS suspension, PLGA/PEG MS suspension, PLGA NM suspension, PLGA/PEG NM suspension, PLGA MS tablet, PLGA/PEG MS tablet, PLGA NM tablet and PLGA/PEG NM tablet.

### Example 3

### Characterization of Microparticles

The size and morphology of microparticles were examined using a scanning electron microscope (SEM; 7401 F, Jeol, Japan). To determine the size distribution of microparticles, the microparticles were assessed with a Coulter counter (Multisizer 4, Beckman Coulter, CA, USA) equipped with a 50-μm aperture. To examine the increase in surface area of the NM, both MS and NM were examined with a surface area and porosity analyzer (TriStar II 3020, Micromeritics, Ga., USA). The surface area was measured with the CO₂ adsorption/desorption method over a relative pressure range of P/P0=0.01-0.025 at 0° C. and calculated, using the Dubinin-Astakhov model.29. The samples were degassed for >72 h at room temperature before measurement. Gel permeation chromatography (GPC) was performed to determine the presence of PEG in the microparticles. 31, 32 Thus, the microparticles were dissolved in THF and filtered through a 0.2 μm-pore membrane filter (Whatman, Clifton, N.J., USA), which was then analyzed by high performance liquid chromatograph (HPLC; Waters 515, Waters, MA, USA) at a flow rate of 1.0 ml/min through three columns in series (PLgel 5.0 guard; 50 mm×7.5 mm, MIXED-C; 300 mm×7.5 mm and MIXED-D; 300 mm×7.5 mm, Polymer Laboratories, Shrewbury, UK) with THF as eluent at 35° C. The GPC system was calibrated with polystyrene standards before use. Thermogravimetric analysis (TGA; TGA-Q50, TA Instruments, DE, USA) was performed to further confirm the presence of PEG in the microparticles. A known amount of the microparticles (20-30 mg) was placed in a platinum pan under nitrogen gas flow, where the temperature was increased from 40° C. to 600° C. at a rate of 10° C./min. A powder of intact PLGA and PEG was also measured for comparison.

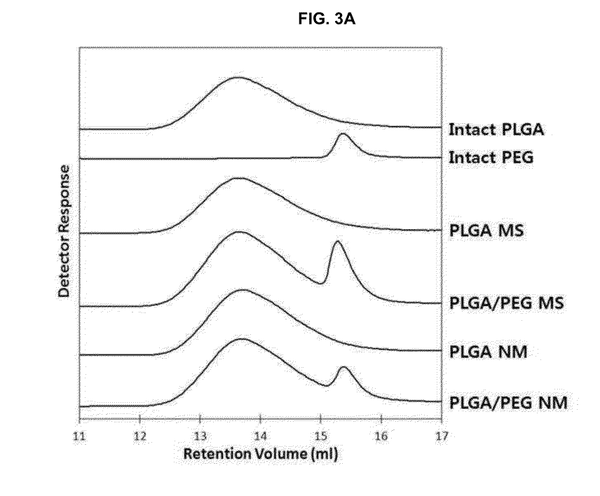

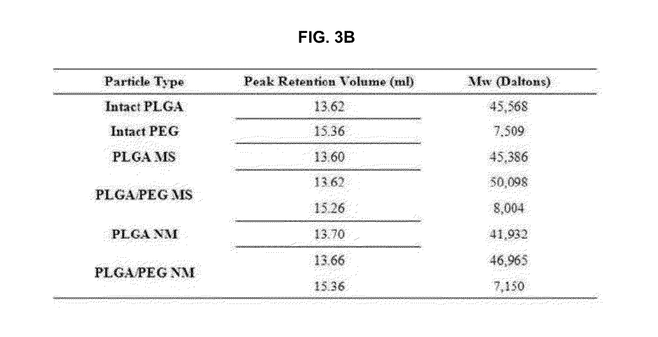

Results are shown in FIGS. 3, 4 and 5. When the gel permeation chromatography (GPC) analyses were performed to validate the presence of PEG in the microparticles, the peak retention volumes of the intact PLGA and PEG powders were observed around at 13.6 ml and 15.3 ml, respectively.

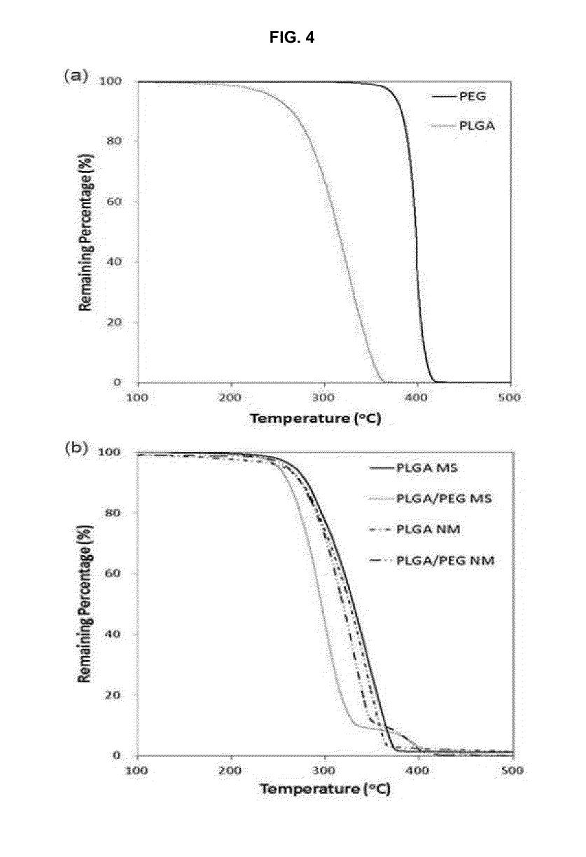

For the microparticles made of PLGA only (i.e., PLGA MS and PLGA NM), a single peak retention volume was observed, as with intact PLGA. On the other hand, the microparticles made of a blend of PLGA and PEG (i.e., PLGA/PEG MS and PLGA/PEG NM) exhibited two distinct peak retention volumes, each originated from PLGA and PEG, respectively. The result from thermogravimetric analyses (TGA) further confirmed the presence of PEG in PLGA/PEG MS and PLGA/PEG NM (FIG. 4). For intact PLGA and PEG, the weight losses by decomposition were observed at 130° C.-370° C. and 305° C.-415° C., respectively (FIG. 4A). The microparticles composed of PLGA only (i.e., PLGA MS and PLGA NM) exhibited a single weight loss in the same temperature range, as with intact PLGA. However, PLGA/PEG MS and PLGA/PEG NM exhibited two consecutive weight losses due to the presence of both PLGA and PEG (FIG. 4b). According to the second weight loss at 355° C.-410° C., both PLGA/PEG MS and PLGA/PEG NM were suggested to contain a similar amount of PEG of about 10 wt %.

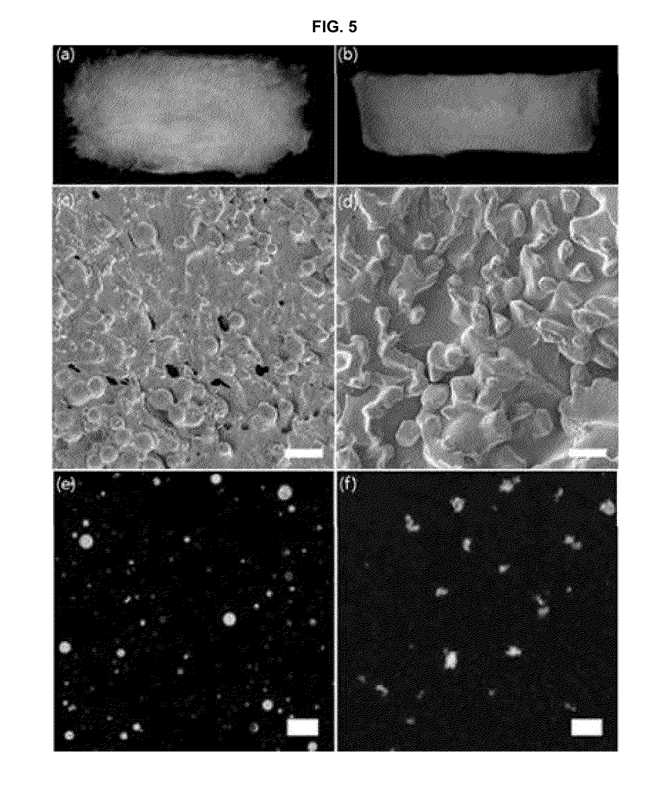

In FIG. 5, a and b show the fluorescence images of a tablet, where the bright signals indicated the presence of Nile-Red loaded microparticles. The tablet was 3 mm in width, 6 mm in length and 2 mm in height with an equivalent volume of approximately 30 μl, which was similar to the volume of a single-dose eye drop. In FIG. 5, c and d shows the SEM images of the tablet surfaces, where the microparticles were seen to be bound with the polymeric medium, PVA. When immersed in pH 7.4 PBS, a porous tablet medium dissolved away rapidly (<1 min), freeing the fluorescent microparticles in the media (FIG. 5, e and f). The distinctive shapes of the microparticles, i.e., spherical and nanostructured ones, were discernible, depending on their own geometry. The agglomerated nanofibers in each of the NM were not seen to be disassembled in aqueous media.

Also, Table 1 below shows the specific surface area which was measured with the CO₂ adsorption/desorption method over a relative pressure range of P/P0=0.01-0.025 at 0° C. and calculated, using the Dubinin-Astakhov model . As shown in Table 1, the increase in specific surface area of the NM (8.13 m²/g) was apparent, showing a more than 13-fold increase, as compared with that of the MS (108.78 m²/g). Such increase in the specific surface area results in the increase in the friction of NM on preocular surface, thereby improving the retention time of NM in the eye.

TABLE 1

| Particle Type | Specific Surface Area (m²/g) |
|---|---|
| PLGA/PEG MS | 8.13 |
| PLGA/PEG NM | 108.78 |

### Example 4

### In vivo Evaluation of Preocular Microparticle Retention

In vivo study was performed with male New Zealand White rabbits (Cheonan Yonam College, Chungheongnam-do, Korea), weighing 3.5-4.5 kg, without any known ocular abnormality. The experiment procedure was approved by the Institutional Animal Care and Use Committee (IACUC No. 10-0304) at Seoul National University Hospital Biomedical Research Institute. The animals were housed singly in a standard cage at controlled temperature (21±1° C.) and humidity (55±1%) with a 12/12-h light-dark cycle without any restriction of food and water.

In vivo preocular retention was tested with the eight different microparticle formulations prepared in Example 2. For administration of the microparticle formulation, either aqueous suspension or a dry tablet, each rabbit was taken out from the cage and positioned in a restrict bag with only the head exposed. Then, the formulation, containing 0.5 mg microparticles, was administered into the lower fornix of the rabbit eye without anesthesia and the eye was manually closed for 3 min. After that, the rabbit was placed back in the cage before sample collection. The rabbits were locally anesthetized (30 μl of Alcaine Eye Drops 0.5%, Alcon, Korea) on the eye and the surface was wiped thoroughly with a surgical sponge (PVA Spears, Network Medical Products, Ripon, UK) 10 min, 30 min, 60 min, 90 min and 120 min after administration of the formulation. Then, the surgical sponge was immersed in acetone and agitated strongly for 1 h to completely extract Nile Red, which was analyzed with fluorescence spectroscopy (FS2, Scinco, Korea) to determine the amount of collected microparticles.

Also the images of the preocular surface of the rabbit after administration of the microparticle formulations were obtained. Before imaging, the rabbit was anesthetized with a subcutaneous injection of a cocktail of 17.5 mg·kg-1 ketamine, 5 mg·kg-1 xylazine and 0.2 mg·kg-1 acepromazine. One additional booster (a half dose of the first injection) was used if necessary. Each of the eight microparticle formulations was administrated as stated above and the fluorescent images of Nile Red-loaded microparticles left on the rabbit eye were obtained 10 min, 30 min, 60 min, 90 min and 120 min after administration. For this, the eye surface was imaged with a camera (HTC raider, HTC, Taiwan) equipped with a Tetramethylrhodamine Isothiocyanate (TRITC) emission filter with transmission wavelengths of 594-646 nm (MF620-52, Thorlabs, NJ, USA) while the eye was illuminated with a LED lamp (AM-R5 mini, Aimai, Korea) equipped with a TRITC excitation filter with transmission wavelengths of 531-551 nm (MF542-20, Thorlabs, NJ, USA). The image included the whole anterior surface of the eyeball and the lower fornix while the upper fornix was not imaged since almost no microparticles were observed.

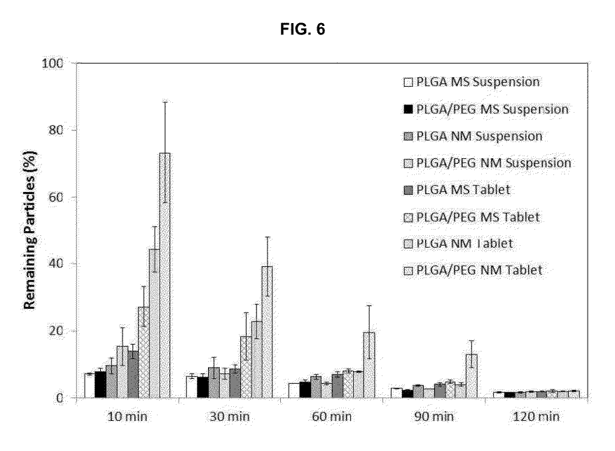

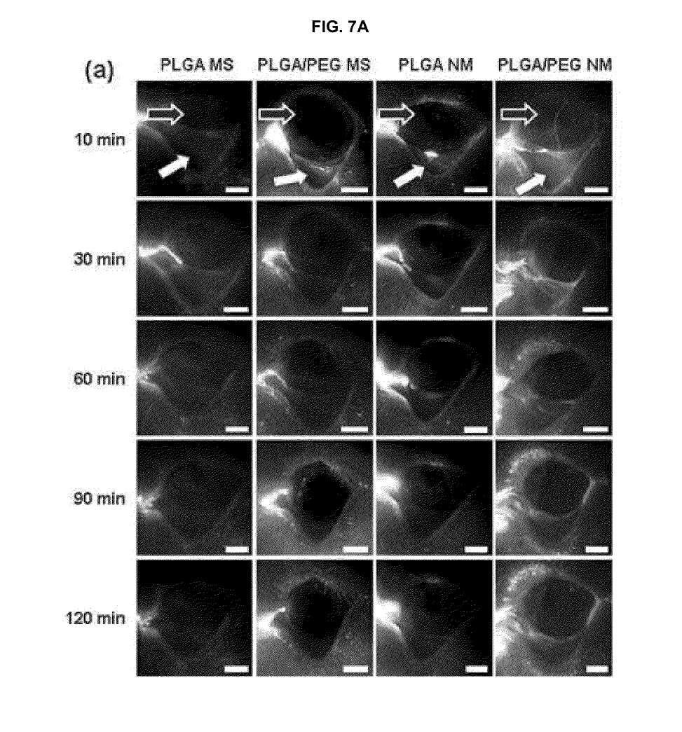

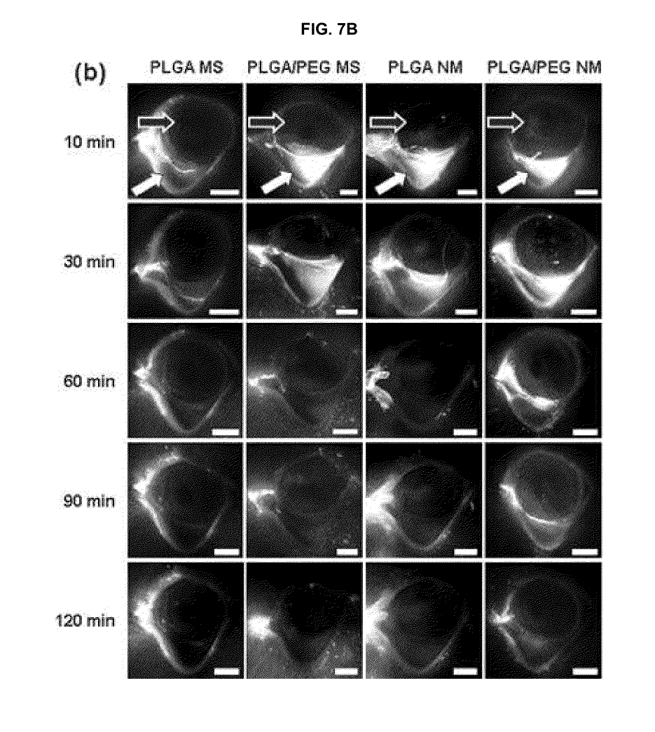

Results are shown in FIGS. 6 and 7. All microparticles in suspension, regardless of their types, exhibited poor preocular retention. That is, only 7-15% of microparticles remained at 10 min and most of the microparticles disappeared from the preocular surface after 30 min. The tablet formulation only could not improve the preocular retention property of the microparticles. The PLGA MS tablet exhibited only 14% and less than 10% of remaining microparticles at 10 min and after 30 min, respectively. When combined with mucoadhesiveness, on the other hand, the effect of tablet formulation was observable. For PLGA/PEG MS tablet, the average percentages of remaining microparticles increased from 8% to 27% and from 6% to 18% at 10 min and 30 min after administration, respectively as compared with PLGA/PEG MS suspension.

The combined effect of nanostructured surface and tablet formulation was also evident. The PLGA NM tablet exhibited a statistically significant increase in preocular retention from 9% to 44% 10 min after administration, as compared with the PLGA NM suspension (p<0.001). This indicates that a rough surface of the NM increases their friction on preocular surface, thereby hindering clearance of the microparticles even without mucoadhesion property. The enlarged surface area might also help to increase the adhesion of the microparticles by van der Waals forces.

Among all the formulations, the best preocular retention was observed with a PLGA/PEG NM tablet. The average percentages of remaining microparticles were 73%, 39%, 19% and 13% at 10 min, 30 min, 60 min and 90 min, respectively. Notably, these dramatic increases in retention were statistically significantly different from all other formulations tested in this work (p<0.05). As compared with the PLGA/PEG NM suspension, the PLGA/PEG NM tablet exhibited 4.8-, 5.5-, 4.5- and 4.8-fold increases in preocular retention at 10 min, 30 min, 60 min and 90 min, respectively. This indicates that the tablet dosage form is effective on improving the mucoadhesion of the PLGA/PEG NM on the eye surface.

Consistent results were obtained in FIGS. 6 and 7, in which it was found that the microparticles' residence on the eye was significantly increase by nanostructures formed or mucoadhesiveness. Particularly, the PLGA/PEG NM tablet exhibited the highest visibility of the microparticles until 30 min after administration. The microparticles were present at the preocular surface for up to 90 min, which disappeared almost completely at 120 min. The microparticles remaining on the preocular surface were found mostly in the lower fornix. This could be ascribed to the fact that the mucin is known to be produced mostly at the lower fornix, thereby higher mucoadhesion.

The results shows the synergy between the increased surface-area resulted from the nanostructure formed on the NM and mucoadhesiveness, would allow better interaction of the NM with the mucous layer of the eye. In contrast, the residence increase in MS was slight. That is, the NM of the present disclosure in tablet formulation became dissolved in tear to release only the microparticles on the preocular surface, while increasing the tear viscosity and thus, allowing more time for microparticle interaction with the mucous layer on the eye.

### Example 5

### In vivo Safety Evaluation

To assess the in vivo safety, after topical administration of a PLGA/PEG NM tablet, the rabbit eye was examined with microscopy and external ophthalmic photography by a professional ophthalmologist. The intraocular pressure was also monitored, using a tonometer (Tonopen AVIA, Reichert, NY, USA). For each of the animals, the left eye was treated with a PLGA/PEG NM tablet and evaluated 1 h, 2 h and 24 h after administration while the right eye remained intact. Four rabbits were tested for safety evaluation in this work.

Cell toxicity was tested using TOX7 Kit (Sigma-Aldrich, USA) following the manufacturer's instruction. Briefly, L929 Fibroblast (Korea Cell line bank, Korea) were seed onto each well of 6 well plate at 5×10⁵ cells/well and each well was treated with two tablets of NM of the present disclosure followed by incubation for 24 h at 37° C. After the incubation, the media were removed from the plate, which was then centrifuged for 4 min at 250×g. The 50 μl of the supernatant was used for the test using TOX7 kit. Each number represents a result from 4 independent experiments, and as a positive control, 1% Triton-X100 (Sigma, MO, USA) was used.

Cell toxicity was calculated using the following formula:

Cell toxicity(%)=(Tablet sample-release background)/(positive control-release background)×100%

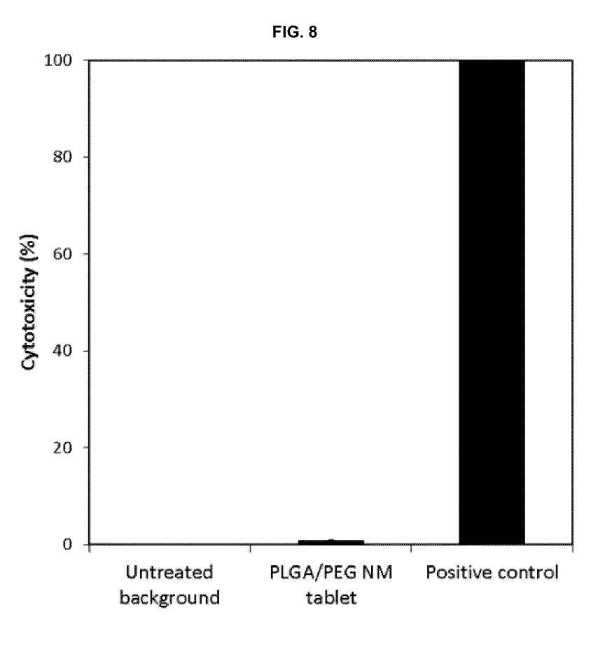

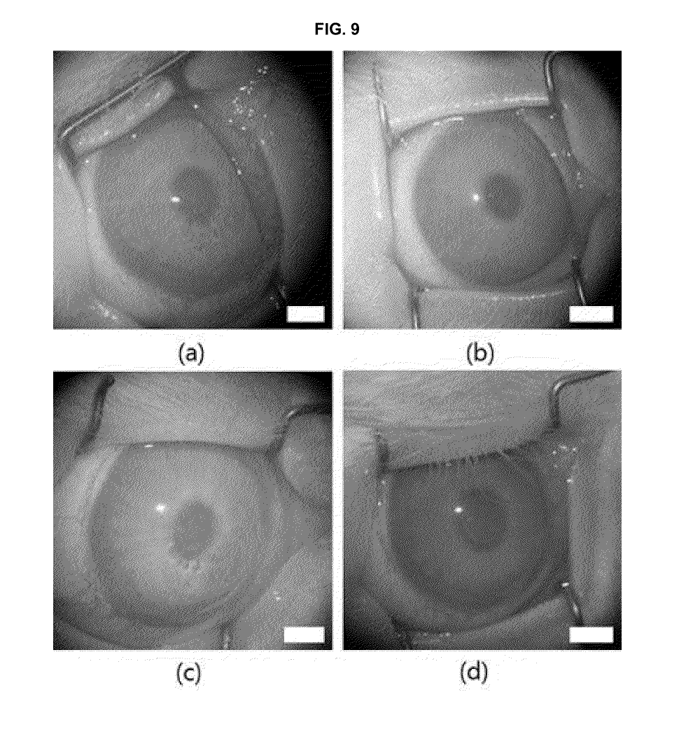

Results are shown in FIGS. 8 and 9. As shown in FIG. 8, it was found that the present PLGA/PEG NM tablet is safe without cytotoxicity. FIG. 9 is a result of safety test of PLGA/PEG tablet of the present disclosure administered on the rabbit eye. During the test, no significant side effect was found except a minor conjunctival injection. Conjunctivitis was found in the normal eye that was left untreated, which was thought to result from the dryness of the eye left open during the anesthesia. The Intra ocular pressure (IOP) was not significantly changed after the administration of PLGA/PEG NM tablet. The IOP measurement was 16.8±1.4 mmHg, which is normal value for the rabbit.

Statistical Analysis

The percentage of the microparticles remaining on the preocular surface of the rabbit was calculated based on the amount of the microparticles initially applied to the eye (i.e., 0.5 mg microparticles per dose). Mean percentages of remaining microparticles among the eight different microparticle formulations were analyzed for statistical significance with ANOVA with α=0.05, followed by pairwise comparisons using a Tukey's post hoc test.

### Example 6

### Test of Drug Release Using microparticles

6-1. BRT loading and Release

Although currently widely used glaucoma drug “Alphagan eye drops” comprising Brimonidine Tartrate 0.15% is highly effective in treating glaucoma, it is inconvenient that it requires to be administered 3 times a day.

In this Example, PLGA MS, PLGA/PEG MS, PLGA NM, PLGA/PEG NM loaded with the same amount of Brimonidine Tartrate(BRT) (Nanjing Yuance Industry & Trade Co., Ltd, Nanjing, China) as contained in Alphagan were prepared and formulated into tablets as Example 2. For PLGA/PEG MS, PLGA 500 mg, PEG 625 mg (125%) Brimonidine 50 mg was dissolved in 5 ml of DCM and sonicated for 2 min at 160 W for mixing. The resulting solution was then added to 50 ml of PVA 1% (20 mM phosphate buffer at pH=12) and sonicated for 30 sec at 100W followed by stirring at 100 rpm under vacuum (−12.5 psi) for 40 min to evaporate DCM. Then the suspension was centrifuged for 10 min at 3500 rpm to separate the particles, which were then freeze dried. PLGA MS was prepared by the same method as described above except that PEG was not used. In the process, 625 mg of PEG which is 125% relative to PLGA was used, most of the PEG used was removed during the preparation process and only 6% of PEG compared to PLGA was found to be present in the microparticles based on NMR data.

Brimonidine is easily dissolved in water and thus MS is generally prepared using double emulsion methods. In the present case, drug and PEG were loaded to PLGA (wall material) by mixing PLGA (wall material), PEG (mucoadhesive promotor) and Brimonidine in organic solvent DCM followed by a sonification. In this way, Brimonidine which is not dissolved in DCM is physically loaded to PLGA.

For preparing PLGA/PEG NM, to 3.35 ml of solvent (DCM:THF: DMF=3:1:1, v/v/v), 1 g of PLGA 1 g, 60 mg of PEG and 50 mg of Brimonidine were added, which was then electrospun under tip-to-collector distance=10 cm; collector rotation=100rpm; Needle gauge 26G; and Feeding rate=3.0 ml/h. Freeze milling was performed for 30 min at 14 CPS (14 back and forth movements per sec). As with MS, PEG was contained 6% compared to PLGA in the final product. PLGA NM was prepared by the same method as described above except that PEG was not used. With the use of electrospinning methods, it was found that no loss of drug and PEG was resulted (data not shown). The prepared microparticles are summarized in Table 2a.

For the analysis of specific surface area, the same method as in Example 3 was used except that N2 was used instead of CO2. Thus the absolute values are different; however, the differences are identical as 13 times as in Table 2b.

TABLE 2a

| Microparticle | PEG weight percent | Mean size | Brimonidine amount | Microparticle amount |
|---|---|---|---|---|
| Type | (wt %) | (μm) | (μm/mg) | in tablet (mg) |
| PLGA MS | 0% | 1.59 | 21.6 | 2.43 |
| PLGA/PEG MS | 5.91% | 1.60 | 20.1 | 2.61 |
| PLGA NM | 0% | 1.60 | 22.6 | 2.32 |
| PLGA/PEG NM | 5.93% | 1.66 | 23.5 | 2.34 |

TABLE 2b

| Microparticle Type | Specific surface area (m²/g) |
|---|---|
| PLGA/PEG MS | 3.211 |
| PLGA/PEG NM | 44.216 |

As negative control, tablet (PVA tablet) without MS and NM, containing only Brimonidine Tartrate was used.

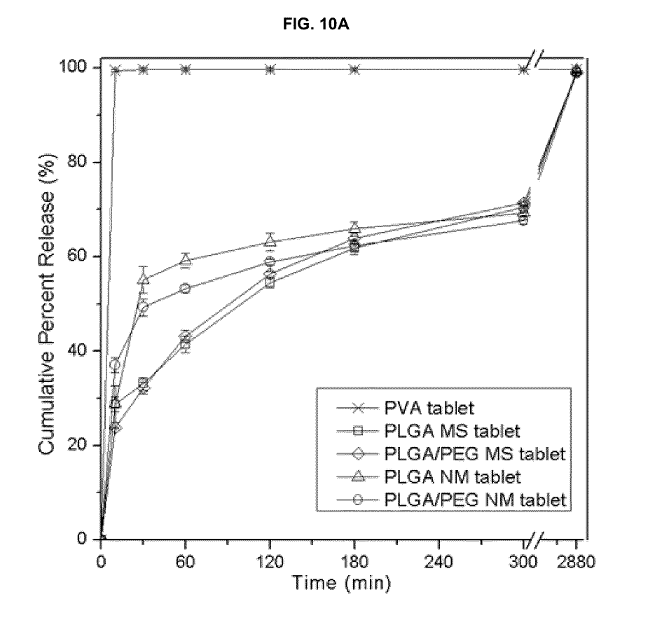

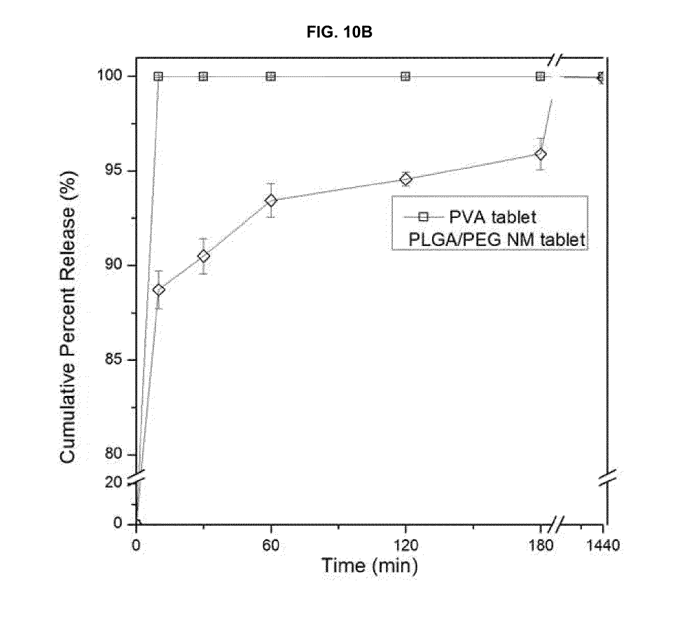

MS and NM as prepared above were used for in vitro drug release test. One tablet prepared with microparticles was immersed in 10 ml pH 7.4 PBS buffer, from which 1 ml of sample was collected over time as indicated in FIG. 10 for HPLC analysis under the following condition: HPLC (agilent 1260 seires, Agilent technologies, USA)(Column=Poroshell 120 EC-C18, 4.6×100 mm, 2.7 um, Mobile phase=20 mM phosphate buffer (pH=2.5) 80% and 20% Methanol, Flow rate=1 ml/min. 10 μl injection, 248 nm, Temp=40° C.)

As shown in FIG. 10a, PVA tablet, a porous material, which does not contain microparticles rapidly released drug in the buffer and 99% of the drug loaded was found to be released within the first 10 min from the PVA tablet. Regardless of the presence of PEG, MS showed an initial burst of release in which about 23-30% of the drug loaded was released within the first 10 min, and about 70% of the drug was released over 300 min in a sustained manner. In the case of NM, it showed an initial burst of release in which about 28-80%, which is greater than that of MS, of the drug loaded was released within the first 10 min, and about 70% of the drug was released over 300 min in a sustained manner. This indicates that NM which has a larger specific surface area than that of MS was able to contact with more amount of buffer than MS, thereby releasing more drug during the initial burst.

### 6-2 Dorzolamide Loading and Release

Using the method as described in Example 6-1, PLGA/PEG NM loaded with 556.5 μg Dorzolamide(Xi'an of natural field bio-technique Co., Ltd, Xi′an, Shaanxi, China) with 6% PEG was prepared by electrospining.

As negative control, tablet (PVA tablet) without NM, containing only Dorzolamide was used.

NM prepared as above used for in vitro drug release test as described in Example 6-1.

As shown in FIG. 10b, as with BRT, more than 99% of the drug loaded was released within the first 10 min from the PVA tablet. For PLGA/PEG NM tablet, 88% of the drug loaded was released for the first 10 min, and 7% of the drug was released during the next 10˜180 min, thus 96% of the drug was released upto 180 min.

This result indicates that when PLGA/PEG NM is used as a drug delivery vehicle, the retention time on the eye is dramatically increased thereby increasing the amount of drug delivered to the eye. Thus the number of administration per day can be reduced from twice or three times a day to once a day or once per several days to achieve the same or even increased efficacy compared to the conventional delivery system.

### Example 7

### Therapeutic Effect by the Drug Delivered using the Microparticles

The formulation of microparticles as prepared in Examples 6-1 and 6-2 was administered to the rabbit eye according to Example 4 and IOP was measured as described in Example 5.

For testing therapeutic effect by BRT, a total of 7 samples were used. Alphagan P is a brimonidine formulation which is in current clinical use and contains purite as a preservative, by which the absorption of brimonidine is known to be increased. 35 μl of Alphagan P was used in which 52.5 μg brimonidine is contained. That is, 35 μl of alphagan P, 35 μl of Brimonidine, PVA tablet (just containing 52.5 μg of brimonidine without any microparticles) and four different types of microparticles prepared as described above containing 52.5 μg of brimonidine were used for the test. For testing therapeutic effect by dorzolamide, 25 μl of Trusopt 2% (MSD, USA), PVA tablet (just containing 556.5 μg of dorzolamide 556.5 μg without any microparticles) and four different types of microparticles prepared as described above containing 556.5 μg dorzolamide were used for the test.

Then, a device for measuring IOP called Tonopen as described in Example 5 was used to measure IOP before the administration and 0.5 h˜13 h after the administration to confirm the efficacy of the drug.

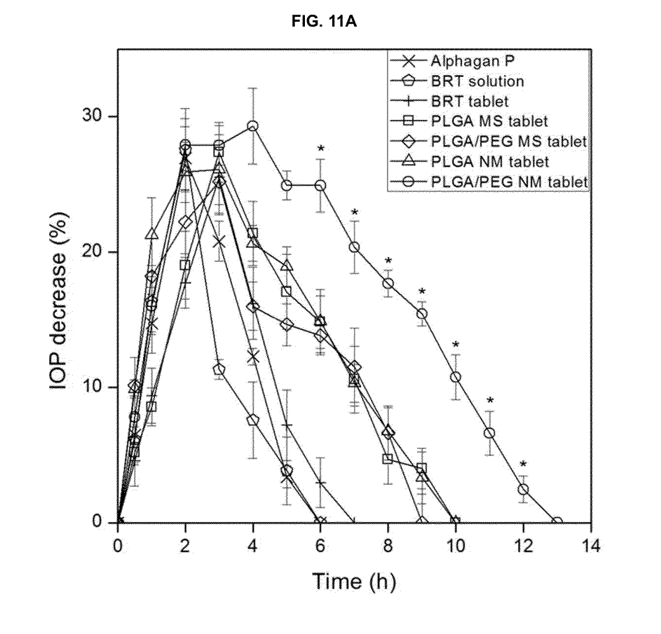

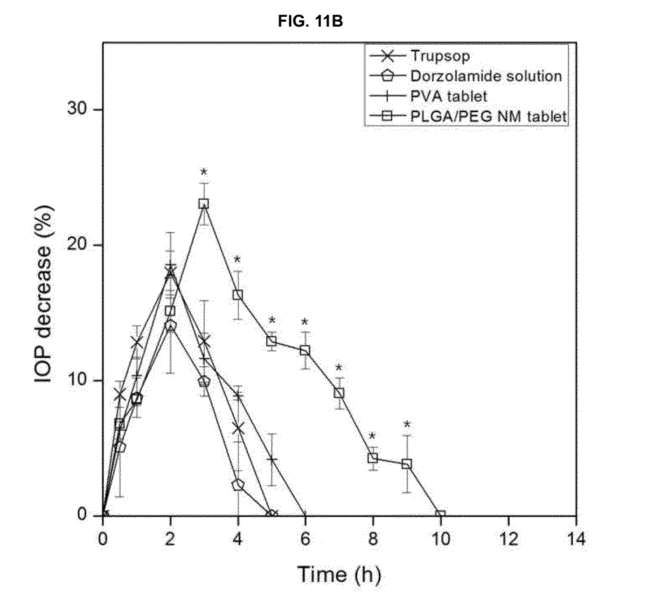

Results are shown in FIGS. 11a and 11b.

As shown in FIG. 11a, Alphagan P and brimonidine was effective for 6 hours at the maximum and PVA only was effective for 7 hours at the maximum. For PVA tablets loaded with PLGA MS, PLGA/PEG MS, or PLGA NM, the drug was delivered by majority of the microparticles residing in preocular region in a sustained manner and thus was effective for 9˜10 hours. The PLGA/PEG NM which exhibited the best mucoadhesiveness and thus the longest sustained release of the drug released was effective upto 13 hours after the administration. Statistically significant decrease in IOP was observed at 6, 7, 8, 9, 10, 11 and 12 hours by treatment with PLGA/PEG NM tablet compared to control and other formulations (p<0.05).

In FIG. 11b, Trusopt and Dorzolamide solution was effective for 5 hours in decreasing IOP, and PVA was effective for 6 hours. In contrast, the same amount of dorzolamide was delivered using the PLGA/PEG NM tablet prepared, the decrease in IOP was observed upto 10 hours. Statistically significant decrease in IOP was observed at 3, 4, 5, 6, 7, 8 and 9 hours by treatment with PLGA/PEG NM tablet compared to control and other formulations (p<0.05).

These results indicate that the present formulation can be advantageously used as an effective vehicle for sustained delivery of various drug, thereby maximizing the therapeutic effect exerted by the drug.

### Example 8

### Pharmacokinetic Analysis

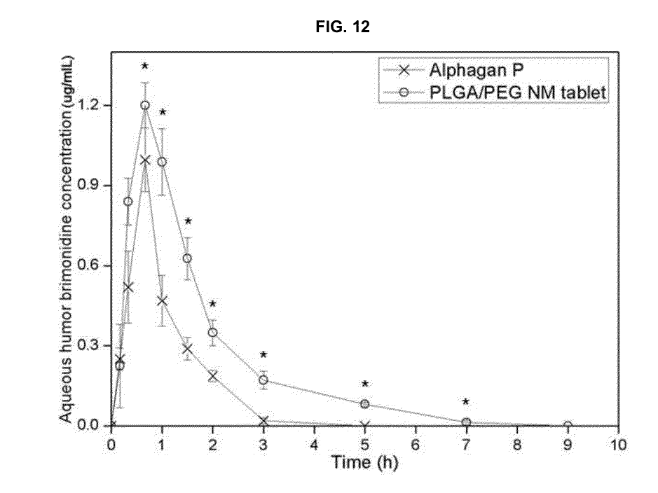

Seven different formulations loaded with brimonidine prepared as described in Example 6-1 were topically administered to the rabbit eye and pharmacokinetics of the drug administered was examined as described below from the aqueous humor taken at a specified time in FIG. 12 after the administration. Specifically, the rabbit was generally anesthetized as described in Example 4 at the specified time after the administration and 50 μl of Aqueous humor (AH) was obtained from the rabbit eye using 1 ml syringe with 30 gauge needle, which was then used for SPE (Solid Phase Extraction) and HPLC analysis. First, Brimonidine Stock solution was prepared at the final concentration of 2000 μl/ml in distilled water, from which 2000, 1000, 800, 600, 400, 200, 100, and 50 μg/ml of Brimonidine solution were prepared. For the calibration, 25 μl of AH from no treatment control, 25 μl Stock Solution (2000, 1000, 800, 600, 400, 200, 100, 50 μg/ml), 50 μl IS (Internal standard, clonidine)(50 μg/ml) and 200 μl of PBS (pH=8) were mixed and centrifuged at 11000×g for 10 min. and 100 μl of the supernatant was used for SPE analysis.

For the sample from the rabbit eye treated with the drug, 50 μl of AH, 50 μl of IS (50 μg/ml) and 200 μl of PBS (pH=8) were mixed and centrifuged at 11000×g for 10 min and 100 μl of the supernatant was used for SPE analysis. SPE (Bond-Eluct-C18, 100 mg, 1 ml, Agilent Technologies) analysis was proceed as below. Before the sample injection, it was washed with MeOH (1 ml) and distilled water (1 ml). Each sample for the calibration and for the analysis was then injected and washed with 1 ml of PBS and eluted with 1 ml of 70% acetonitrile, followed by evaporation for 3 hrs at 40° C. using SpeedVac (Thermo Savant SPD 2010 SpeedVac System, Thermo Electron Corporation, USA). After the evaporation, 100 μl of water was added to the sample, which was then filtered (0.2 μm, 4 mm, Whatman, UK) and used for HPLC analysis.

HPLC (Agilent 1260 Series, Agilent Technologies, CA, USA) was used for determining the concentration, in which 50 μl of sample was injected and analyzed under the following condition: Mobile phase=20 mM phosphate buffer (pH=2.5) : MeOH=9:1, flow rate 1 ml/min, 248 nm

Results are shown in Table 3 and FIG. 12, which shows the concentration of brimonidine in AH after administration of each formulation. Cmax (maximum concentration) was found highest in PLGA/PEG NM tablet as 1.201 μg/ml. Tmax (time at Cmax) was found to be identical in all samples as 40 min (0.667 hour). Area under curve (AUC) indicating a drug absorption rate was found to be 1.013 for Alphagan P, 0.795 and 1.136 for BRT solution without purite and BRT tablet, respectively, and 1.3˜1.4 for PLGA MS, PLGA/PEG MS and PLGA/NM tablets. PLGA/PEG NM was found to have the highest AUC of 2.078.

TABLE 3

| Formulation | Cmax (μg/mL) | tₘₐₓ (hr) | AUC (μg · h/mL) |
|---|---|---|---|
| Alphagan P | 0.997 | 0.667 | 1.013 |
| BRT solution | 0.692 | 0.667 | 0.795 |
| BRT tablet | 0.793 | 0.667 | 1.136 |
| PLGA MS tablet | 0.834 | 0.667 | 1.300 |
| PLGA/PEG MS tablet | 0.910 | 0.667 | 1.388 |
| PLGA NM tablet | 1.004 | 0.667 | 1.380 |
| PLGA/PEG NM tablet | 1.201 | 0.667 | 2.078 |

### Example 9

### Adhesiveness Test of the Microparticles on the Mucous Membrane of Intestine.

MS and NM loaded with Nile Red prepared as described in Example 1, and Nile red was suspended in PBS pH.7.4 at the final concentration of 1.8 pg/ml Nile Red. The mucous membrane of the rabbit was placed facing upward and 2 drops of the suspension prepared (25 μl) was applied to the mucous membrane and incubated for 5 min.

Then, the treated mucous membrane was washed in 100 ml of PBS pH 7.4 by dipping in and out for 20 times, after which images were taken using TRITC filter and camera to confirm the residual amount of Nile Red, MS and

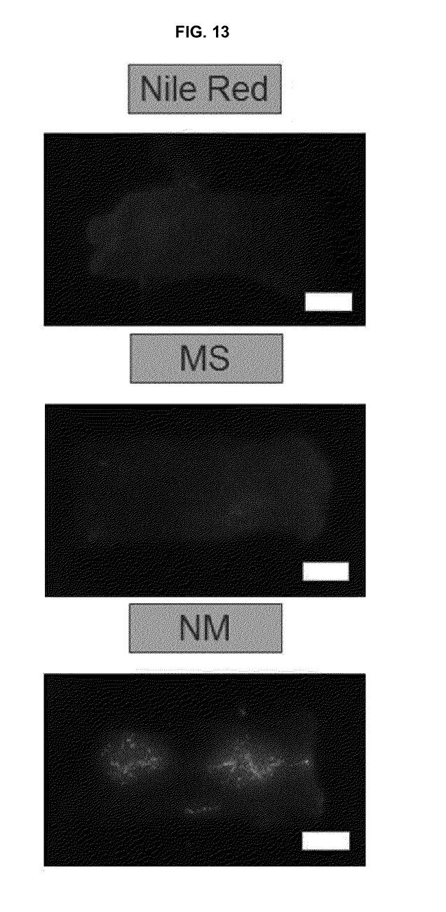

NM. Results are shown in FIG. 13.

FIG. 13 is.fluorescence images of Nile Red and MS and NM loaded with Nile Red. As shown in FIG. 13, the intestine applied with MS and Nile Red, it was found that almost no Nile Red and MS were left after washing 20 times in contrast to the result from NM treated intestine, in which large amount of NM was found to be left on the intestine after the washing. These results indicate that the relatively large specific surface area of NM has increased its adhesiveness to the mucous membrane in the intestine thereby improving its retention time compared to MS. Thus it also indicates that the present NM can be advantageously used as a drug delivery vehicle for sustained release of the drug in a variety of mucous membranes thereby maximizing the therapeutic efficacy of the drug administered.

While the present device has been shown and described in terms of various aspects, it will be apparent to those skilled in the art that various modification and changes may be made without departing the principles and spirit of the invention. Thus the scope of the invention must be defined by the appended claims and their equivalents.

With respect to the use of substantially any plural and/or singular terms herein, those having skill in the art can translate from the plural to the singular and/or form the singular to the plural as is appropriate to the context and/or application. The various singular/plural permutations may be expressly set forth herein for sake of clarity.

1-12. (canceled)

13. A nanostructured mucoadhesive microparticle comprising a biocompatible adhesive agent, the microparticle having a surface with a nanostructure formed thereon thereby having an enlarged specific surface area and an increased adhesiveness to a mucous membrane.

14. The microparticle of claim 13, further comprising a diffusion control material.

15. The microparticle of claim 13, wherein the mucous membrane is an ocular, pulmonary, buccal, bronchial, endometrium, esophageal, olfactory, penile, vocal, sublingual, rectal, gastric, intestinal, colonic, oral, nasal, anal, or vaginal mucous membrane.

16. The microparticle of claim 13 in the form of tablet.

17. The microparticle of claim 13, wherein the biocompatible adhesive agent is a water soluble polymer.

18. The microparticle of claim 17, wherein the water soluble polymer is a water-soluble synthetic polymer.

19. The microparticle of claim 18, wherein the water-soluble synthetic polymer is a polyethylene glycol (PEG).

20. The microparticle of claim 14, wherein the diffusion control agent is a polylactide, a polyglycolide, a poly(lactic-co-glycolic acid) (PLGA), a polyorthoester, a polyanhydride, a poly(amino acid), a poly(hydroxybutyric acid), a polycaprolactone, a polyalkylcarbonate, an ethylcellulose, a chitosan, a starch, a guar gum, a gelatin, a collagen, or a combination thereof.

21. The microparticle of claim 14, wherein the biocompatible adhesive agent is a PEG and the diffusion control agent is a PLGA.

22. The microparticle of claim 13, wherein the nanostructure is formed on the surface of microparticle by electrospinning and freeze-milling the biocompatible adhesive agent.

23. The microparticle of claim 13, wherein the microparticle further comprises a small molecule, a protein drug, a radionuclide, a nucleic acid based drug or a combination thereof for a sustained release thereof through the adhesiveness to a mucous membrane.

24. The microparticle of claim 13, wherein the mucous membrane is an ocular mucous membrane, and the microparticle further comprises a therapeutic agent for treating ocular disease selected from the group consisting of an antiviral agent, an antibacterial agent, an anti-fungal agent, an antiallergic agent, an nonsteroidal anti-inflammatory agent, an anti-inflammatory agent, an anti-inflammatory-analgesic agent, an anti-inflammatory enzyme agent, an antibiotic, a sulfa agent, a synthetic penicillin, a therapeutic agent for treating glaucoma, a therapeutic agent for treating cataract, a miotic, a mydriatic, a topical astringent, a vasoconstrictor, an agent for preventing rise of intraocular pressure, a therapeutic agent for treating ocular hypertension, a topical anesthetic, an α1-blocker, a β-blocker, a β1-blocker, a carbonic anhydrase inhibitor, a topical selective H1-blocker, an adrenal cortical hormone, a vitamin B12, a coenzyme type vitamin B2, an anticholinesterase agent, and an organic iodine preparation.

25. A mucoadhesive system for drug delivery comprising the nanostructured mucoadhesive microparticle according to claim 13.

26. The mucoadhesive system of claim 25, wherein the drug delivery is a sustained drug delivery.
